(19) United States
(12) Patent Application Publication
(10) Pub. No.: US 2004/0214883 A1
(43) Pub. Date: Oct. 28, 2004

(54) Calcium monoaspartate

(76) Inventors: Brent P. Hagen, Brigham City, UT (US); Wayne E. White, Thousand Oaks, CA (US); Shane R. Shupe, Harrisville, UT (US)

Correspondence Address: Thompson E. Fehr, Goldenwest Corporate Center, Suite 300, 5025 Adams Avenue, Ogden, UT, 84403, US

(21) Appl. No.: 10/425,159

(22) Filed: Apr. 28, 2003

(51) Int. Cl. A61K 31/325

(52) U.S. Cl. 514/476

(57) ABSTRACT

A stable complex of calcium and aspartic acid having the formula CaCOOCH₂CHNH₂COO, which is highly soluble both in water, making the compound beneficial as a dietary supplement for calcium. One form of the commercial product of the compound has the calcium monoaspartate dissolved in water.

### BACKGROUND OF THE INVENTION

1. Field of the Invention

This invention relates to a compound for incorporating calcium within an animal, including a human being.

2. Description of the Related Art

Nine patents and one published patent application are known to disclose calcium amino acid chelates or calcium aspartate. The patents are U.S. Pat. Nos. 4,738,856; 5,143,732; 5,504,055; 5,516,925; 5,824,353; 6,054,119; 6,159,530; 6,197,815; and 6,207,204. The published patent application is 2002/0025360. Such compounds are, however, distinct from that of the present invention. In particular, those that disclose calcium aspartate do not disclose calcium monoaspartate.

### BRIEF SUMMARY OF THE INVENTION

The compound that is the subject of the present invention is a stable complex of calcium and aspartic acid, which has been determined to be highly soluble in water and, in fact, in both neutral and acid solutions. Such solubility combined with a relatively high content of calcium makes the compound beneficial as a dietary supplement and for use in fortified foods and beverages where a concentrated, soluble calcium source is desired.

### DETAILED DESCRIPTION OF THE INVENTION

The compound of the present invention is calcium monoaspartate.

The formula for this compound is CaCOOCH₂CHNH₂COO.

Since it is highly soluble in water, it is, as mentioned above, beneficial as a dietary supplement. Also, it is convenient, but not necessary, to dissolve the compound in water in order to create a commercial form of the compound.

Examples of methods for making the calcium monoaspartate are as follows:

### EXAMPLE 1

Calcium carbonate was suspended in water to make a 40 percent, by weight, calcium carbonate slurry. An equimolar quantity of 1-aspartic acid was added and reacted with the calcium carbonate in the slurry. The product was oven dried.

### EXAMPLE 2

L-aspartic acid was dissolved in water. Calcium hydroxide was added to this solution in portions to a 1:1 molar ratio. The reaction was allowed to proceed to completion, and the product was oven dried.

We claim:

1. A composition of matter having the formula:
CaCOOCH₂CHNH₂COO.

2. The composition of matter of claim 1, further comprising:
water in which the CaCOOCH₂CHNH₂COO has been dissolved.
